(19) United States
(12) Patent Application Publication
(10) Pub. No.: US 2026/0325699 A1
(43) Pub. Date: Oct. 22, 2026

(54) PHOTOCATALYST MODULE

(71) Applicant: SHARP KABUSHIKI KAISHA, Sakai City (JP)

(72) Inventor: Kazuya IWASE, Sakai City (JP)

(21) Appl. No.: 19/689,861

(22) Filed: May 27, 2026

(30) Foreign Application Priority Data
Mar. 28, 2025 (JP) 2025-056164

(51) Int. Cl. A61L 9/18; B01D 53/00; B01D 53/34; B01D 53/86; B01D 53/88

(52) U.S. Cl. CPC A61L 9/18; B01D 53/007; B01D 53/346; B01D 53/8687; B01D 53/885; A61L 2209/111; A61L 2209/12; B01D 2255/802; B01D 2257/90; B01D 2258/06

(57) ABSTRACT

The photocatalyst module according to the present disclosure includes: a housing; a photocatalyst portion disposed inside the housing; a light source unit provided to irradiate the photocatalyst portion with light; an illuminance sensor provided to detect brightness outside the housing; and a control unit, wherein the housing is provided with a window, and the control unit is provided to switch the window between a light-shielding state for shielding external light entering the housing and a light-collecting state for allowing external light to enter the housing on the basis of an output from the illuminance sensor.

### INCORPORATION BY REFERENCE

This application is based upon and claims the benefit of priority from the corresponding Japanese Patent Application No. 2025-56164 filed on Mar. 28, 2025, the entire contents of which are incorporated herein by reference.

### BACKGROUND

The present disclosure relates to a photocatalyst module.

A photocatalyst device for disinfecting and deodorizing air has been known (e.g., see JP 2022-180816 A). The photocatalyst device described above causes a photocatalyst portion to exhibit photocatalytic activity by emission of light from a light source unit to remove organic substances in the air.

### SUMMARY

The conventional photocatalyst device needs to emit light to the photocatalyst portion from the light source unit to remove organic substances in the air, and thus consumes a large amount of electric power.

The present disclosure has been accomplished in view of the above circumstances, and provides a photocatalyst module installable in a place that needs to be dark during the night with the power consumption being reduced.

The present disclosure provides a photocatalyst module including: a housing; a photocatalyst portion disposed inside the housing; a light source unit provided to irradiate the photocatalyst portion with light; an illuminance sensor provided to detect brightness outside the housing; and a control unit, wherein the housing is provided with a window, and the control unit is provided to switch the window between a light-shielding state for shielding external light entering the housing and a light-collecting state for allowing external light to enter the housing on the basis of an output from the illuminance sensor.

According to the present disclosure, the light-shielding state and the light-collecting state can be switched on the basis of the output from the illuminance sensor. Therefore, when it is sufficiently bright outside the housing, the photocatalyst portion can be irradiated with external light introduced through the window, by which the photocatalyst portion can exhibit photocatalytic activity, and organic substances in the air can be removed. At that time, the light source unit can be turned off or the amount of light emitted from the light source unit can be decreased, whereby power consumption can be reduced.

When it is not sufficiently bright outside the housing, the light source unit emits light toward the photocatalyst portion to cause the photocatalyst portion to exhibit photocatalytic activity, whereby organic substances in the air can be removed. Therefore, even during the night, organic substances in the air can be removed. In this case, the window can be brought into the light-shielding state, and thus, the leakage of light emitted from the light source unit to the outside of the housing can be prevented. Accordingly, the photocatalyst module can be installed in a place such as a bedroom that needs to be dark during the night.

### BRIEF DESCRIPTION OF THE DRAWINGS

FIG. 1 is a schematic cross-sectional view of a photocatalyst module (light-shielding state) according to an embodiment of the present disclosure;

FIG. 2 is a schematic cross-sectional view of the photocatalyst module (light-collecting state) according to the embodiment of the present disclosure;

FIG. 3 is a schematic cross-sectional view of a photocatalyst module (light-shielding state) according to an embodiment of the present disclosure;

FIG. 4 is a schematic cross-sectional view of the photocatalyst module (light-collecting state) according to the embodiment of the present disclosure;

FIG. 5 is a schematic cross-sectional view of a photocatalyst module (light-shielding state) according to an embodiment of the present disclosure;

FIG. 6 is a schematic cross-sectional view of the photocatalyst module (light-collecting state) according to the embodiment of the present disclosure;

FIG. 7 is a schematic cross-sectional view of a photocatalyst module (light-shielding state) according to an embodiment of the present disclosure;

FIG. 8 is a schematic cross-sectional view of the photocatalyst module (light-collecting state) according to the embodiment of the present disclosure;

FIG. 9 is a schematic cross-sectional view of a photocatalyst module (light-shielding state) according to an embodiment of the present disclosure;

FIG. 10 is a schematic cross-sectional view of the photocatalyst module (light-collecting state) according to the embodiment of the present disclosure;

FIG. 11 is a schematic cross-sectional view of a photocatalyst module (light-shielding state) according to an embodiment of the present disclosure;

FIG. 12 is a schematic cross-sectional view of the photocatalyst module (light-collecting state) according to the embodiment of the present disclosure;

FIG. 13 is a schematic cross-sectional view of a photocatalyst module (light-shielding state) according to an embodiment of the present disclosure; and

FIG. 14 is a schematic cross-sectional view of the photocatalyst module (light-collecting state) according to the embodiment of the present disclosure.

### DETAILED DESCRIPTION

The photocatalyst module according to the present disclosure includes: a housing; a photocatalyst portion disposed inside the housing; a light source unit provided to irradiate the photocatalyst portion with light; an illuminance sensor provided to detect brightness outside the housing; and a control unit, wherein the housing is provided with a window, and the control unit is provided to switch the window between a light-shielding state for shielding external light entering the housing and a light-collecting state for allowing external light to enter the housing on the basis of an output from the illuminance sensor.

Preferably, the control unit is provided in such a manner that, when the window is in the light-shielding state, the light source unit irradiates the photocatalyst portion with light, and when the window is in the light-collecting state, the light source unit is turned off or an amount of light emitted from the light source unit is reduced.

Preferably, the housing includes an inlet and an outlet, and the photocatalyst portion is placed at a position where a gas flowing from the inlet to the outlet comes into contact with the photocatalyst portion.

Preferably, the window includes a light-shielding member and a first grid having a plurality of openings, and the control unit is provided to switch the window between the light-collecting state in which the light-shielding member does not cover the openings and the light-shielding state in which the light-shielding member covers the openings by moving the light-shielding member on the basis of the output from the illuminance sensor.

Preferably, the window includes a translucent member that covers the first grid.

Preferably, the first grid includes a plurality of first rods, each of the openings is an opening between two first rods adjacent to each other, and the light source unit is provided to emit light to the photocatalyst portion from the first rods.

Preferably, the light-shielding member is a second grid having a plurality of second rods, and the control unit is provided to switch the window between the light-collecting state in which the second rods do not cover the openings and the light-shielding state in which the second rods cover the openings by moving the light-shielding member on the basis of the output from the illuminance sensor.

Preferably, the second rods include a mirror provided to reflect reflection light resulting from light emitted to the photocatalyst portion from the light source unit.

Preferably, the light source unit is provided to emit light to the photocatalyst portion from the second rods.

Preferably, the window includes a translucent member and a light-shielding member, and the control unit is provided to switch the window between the light-collecting state in which the light-shielding member does not cover the translucent member and the light-shielding state in which the light-shielding member covers the translucent member by moving the light-shielding member on the basis of the output from the illuminance sensor.

Preferably, the light source unit is provided to emit light to the photocatalyst portion from the light-shielding member.

Preferably, the window includes an optical shutter.

The present disclosure will be described in detail below with reference to a plurality of embodiments. The configurations in the drawings and the following description are merely examples and do not limit the scope of the present disclosure.

### First Embodiment

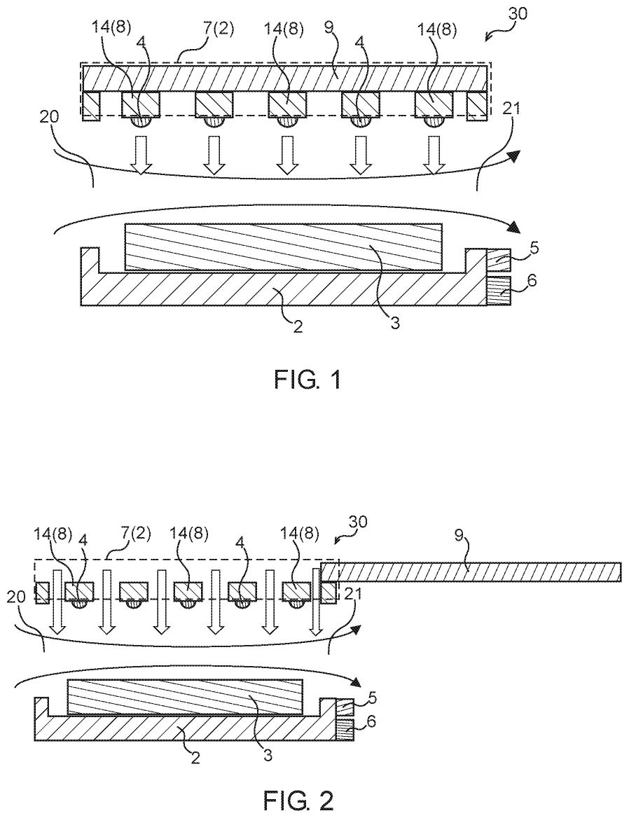

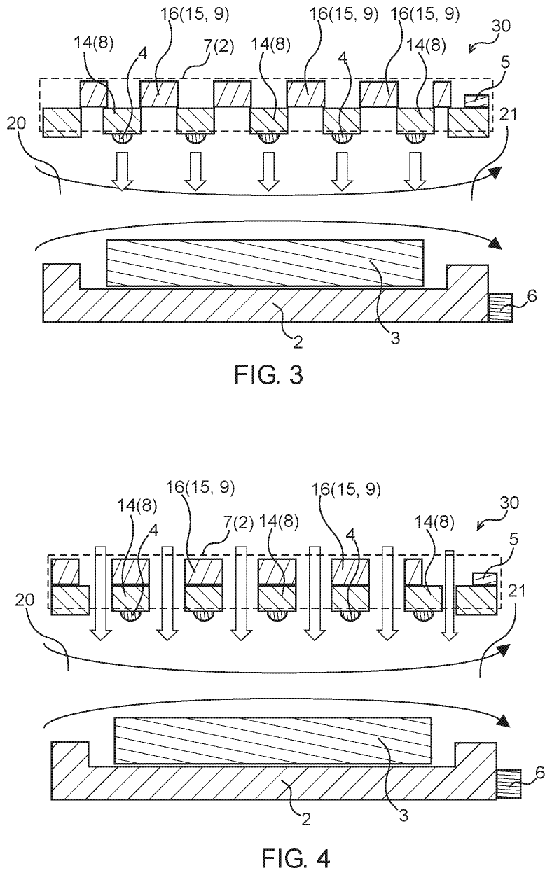

FIGS. 1 and 2 are each a schematic cross-sectional view of a photocatalyst module according to a first embodiment. In FIGS. 1 and 2, a solid arrow indicates a flow of air, and a white arrow indicates the direction of light. The same applies to FIGS. 3 to 14.

A photocatalyst module 30 according to the first embodiment includes a housing 2, a photocatalyst portion 3 disposed inside the housing 2, a light source unit 4 provided to irradiate the photocatalyst portion 3 with light, an illuminance sensor 5 provided to detect brightness outside the housing 2, and a control unit 6. The housing 2 has a window 7. The control unit 6 is provided to switch the window 7 between a light-shielding state for shielding external light entering the housing 2 and a light-collecting state for allowing external light to enter the housing 2 on the basis of an output from the illuminance sensor 5.

The photocatalyst module 30 is a device for removing organic substances such as odor substances in a gas by utilizing photocatalytic activity. The photocatalyst module 30 may be an air purifying device, a deodorizing device, or disinfecting device.

The housing 2 includes the window 7 and houses the photocatalyst portion 3. The housing 2 also includes an inlet 20 and an outlet 21.

The inlet 20 is an opening through which a gas (e.g., air) flows into the housing 2 from the outside of the housing 2. The inlet 20 may have a fan. The inlet 20 may also have a filter. The gas flowing into the housing 2 through the inlet 20 circulates inside the housing 2 and is exhausted to the outside of the housing 2 through the outlet 21. Thus, the gas can be continuously brought into contact with the photocatalyst portion 3 having photocatalytic activity, whereby organic substances such as odor substances can be continuously removed. In addition, the inlet 20 may have a light-shielding structure.

The outlet 21 is an opening through which a gas (e.g., air) is exhausted to the outside of the housing 2 from the inside of the housing 2. In a case where the inlet 20 does not have a fan, the outlet 21 may have a fan. Further, the outlet 21 may have a light-shielding structure.

The light source unit 4 is a section including a light source (e.g., light-emitting element such as a light-emitting diode). The light source unit 4 may be a light source device. The light source unit 4 is, for example, an LED lamp, a fluorescent lamp, an incandescent bulb, a halogen bulb, or a xenon lamp. The light source unit 4 can have a drive circuit (e.g., LED drive circuit). The light source unit 4 can also have a dissipation portion provided to dissipate heat generated in the light source. The light source unit 4 can also have a plurality of light sources. Further, the light source unit 4 can be connected to the control unit 6 so that ON/OFF of the light source is controlled by the control unit 6.

The light source unit 4 is provided to emit light to the photocatalyst portion 3. Thus, organic substances such as odor substances in the gas within the housing can be removed by photocatalytic activity of the photocatalyst portion 3.

The photocatalyst portion 3 is a section containing a photocatalyst (e.g., photocatalytic particles) and is disposed inside the housing 2. The photocatalyst is, for example, tungsten oxide or titanium oxide. The photocatalyst may have cocatalyst particles such as platinum-group metal particles (e.g., Pt, Pd, Rh, Ru, Os, or Ir particles) on the surface thereof. The photocatalyst can exhibit photocatalytic activity by receiving light emitted from the light source unit 4, and due to the photocatalytic activity, the organic substances such as odor substances in the gas flowing through the housing 2 can be removed. In addition, the photocatalyst portion 3 is placed at a position where the gas flowing from the inlet 20 to the outlet 21 comes into contact with the photocatalyst portion 3.

The photocatalyst portion 3 can have a support sheet and a photocatalyst (e.g., photocatalytic particles) supported or fixed on the support sheet. This can increase the probability of the contact of the gas flowing within the housing 2 with the photocatalyst, whereby the organic substances such as odor substances in the gas can be efficiently removed using the photocatalytic activity. The support sheet is, for example, paper, non-woven fabric, or woven fabric. The photocatalyst may be supported on the support sheet or incorporated into the support sheet. The photocatalyst portion 3 may have a pleated-sheet structure.

The illuminance sensor 5 is a sensor provided to detect brightness outside the housing 2 and includes a light-receiving element, for example. The illuminance sensor 5 may include an amplifier circuit that amplifies a current output from the light-receiving element. The illuminance sensor 5 may be connected to the control unit 6 so as to output an output signal (e.g., current) to the control unit 6. The control unit 6 can determine whether the outside of the housing 2 is dark or bright on the basis of the output from the illuminance sensor 5.

The control unit 6 is a section that controls the photocatalyst module 30. The control unit 6 can have a processor (e.g., CPU), a storage unit (e.g., memory), and the like. The control unit 6 may include a drive circuit (e.g., LED drive circuit) for the light source included in the light source unit 4. The control unit 6 may include a signal processing circuit that processes an output signal from the illuminance sensor 5. The control unit 6 may be provided to drive a switching mechanism for the window 7.

The window 7 is a section provided to be capable of collecting external light. The window 7 can be disposed so that the photocatalyst portion 3 is irradiated with external light collected through the window 7. The window 7 can include a switching mechanism provided to switch between the light-shielding state and the light-collecting state. The external light may be sunlight or light emitted from a lighting fixture.

The window 7 included in the photocatalyst module 30 illustrated in FIG. 1 is in the light-shielding state, and the window 7 included in the photocatalyst module 30 illustrated in FIG. 2 is in the light-collecting state.

The window 7 included in the photocatalyst module 30 illustrated in FIGS. 1 and 2 includes a light-shielding member 9 and a first grid 8 that includes a plurality of first rods 14.

The first grid 8 has a configuration in which the first rods 14 are arranged with openings, each of the openings being formed between two first rods 14 adjacent to each other. As illustrated in FIG. 2, external light can be introduced (collected) into the housing 2 through the openings, and the photocatalyst portion 3 can be irradiated with the introduced external light. The width of each of the first rods 14 is 1 cm or more and 5 cm or less, for example. The width of each opening is 1 cm or more and 5 cm or less, for example. The open area ratio of the first grid 8 can be 40% or more and 80% or less, for example.

The light-shielding member 9 has a plate shape and has a size large enough to cover all the openings included in the first grid 8 in the light-shielding state illustrated in FIG. 1.

The window 7 has a switching mechanism provided to be capable of moving the light-shielding member 9 from the state illustrated in FIG. 1 to the state illustrated in FIG. 2 or from the state illustrated in FIG. 2 to the state illustrated in FIG. 1. The switching mechanism can have, for example, a rack (rod-shaped gear) fixed to the light-shielding member 9 and a pinion (cylindrical gear) connected to a motor controlled by the control unit 6. In the switching mechanism, the rack and the pinion can convert rotational motion into linear motion by the rotation of the motor by the control unit 6, by which the light-shielding member 9 can be moved.

The plurality of light-emitting elements of the light source unit 4 included in the photocatalyst module 30 illustrated in FIGS. 1 and 2 are arranged on the plurality of first rods 14 constituting the first grid 8. Further, the plurality of light-emitting elements can be arranged at intervals on each of the plurality of first rods 14. Thus, the entire photocatalyst portion 3 can be efficiently irradiated with light. The interval between the light-emitting elements is 5 cm or more and 20 cm or less, for example.

When it is relatively dark outside the housing 2, the light-shielding member 9 covers the openings of the first grid 8 as illustrated in FIG. 1, so that the window 7 is in the light-shielding state. In this case, the control unit 6 turns on the light source unit 4 so that the photocatalyst portion 3 is irradiated with light. Thus, the photocatalyst portion 3 can exhibit photocatalytic activity, whereby organic substances in the air flowing through the inlet 20 can be decomposed and removed. In addition, since the window 7 is in the light-shielding state, the leakage of light inside the housing 2 can be prevented, and thus, the photocatalyst module 30 can be installed in a place such as a bedroom that needs to be dark.

When the outside of the housing 2 becomes bright and the output from the illuminance sensor 5 exceeds a threshold value, the control unit 6 turns off the light source unit 4 or decreases the amount of light from the light source unit 4, and moves the light-shielding member 9, using the switching mechanism, so as to bring the light-shielding member 9 into a state of not covering the openings of the first grid 8. For example, the control unit 6 changes the photocatalyst module 30 illustrated in FIG. 1 to the photocatalyst module 30 illustrated in FIG. 2.

In this case, external light is introduced into the housing through the openings of the first grid 8, and the photocatalyst portion 3 is irradiated with the introduced external light. Thus, the photocatalyst portion 3 can exhibit photocatalytic activity, whereby organic substances in the air flowing through the inlet 20 can be decomposed and removed. In addition, the light source unit 4 is turned off or the amount of light from the light source unit 4 is decreased, whereby the power consumption of the photocatalyst module 30 can be reduced.

FIG. 3 is a schematic cross-sectional view of a photocatalyst module 30 (window 7 is in the light-shielding state) according to a first modification of the first embodiment, and corresponds to FIG. 1. FIG. 4 is a schematic cross-sectional view of the photocatalyst module 30 (window 7 is in the light-collecting state) according to the first modification of the first embodiment, and corresponds to FIG. 2.

In the first modification, the light-shielding member 9 is a second grid 15 including a plurality of second rods 16. The other configurations are the same as those of the first embodiment described above.

The second grid 15 has a configuration in which the second rods 16 are arranged with openings, each of the openings being formed between two second rods 16 adjacent to each other. Each of the second rods 16 has a width capable of closing the corresponding opening of the first grid 8 in the light-shielding state illustrated in FIG. 3 and overlapping the corresponding first rod 14 in the light-collecting state illustrated in FIG. 4. It is preferable that the width of each of the second rods 16 be substantially equal to the width of each of the first rods 14. Further, it is preferable that the width of each of the second rods 16 be substantially equal to or larger than the width of the opening between the two first rods 14 adjacent to each other.

Since the light-shielding member 9 has a grid shape as described above, the light-shielding state and the light-collecting state can be switched only by slightly moving the light-shielding member 9 as illustrated in FIGS. 3 and 4, and thus, the light-shielding member 9 can be prevented from protruding in the light-collecting state. Therefore, the photocatalyst module 30 can be installed in a narrower area.

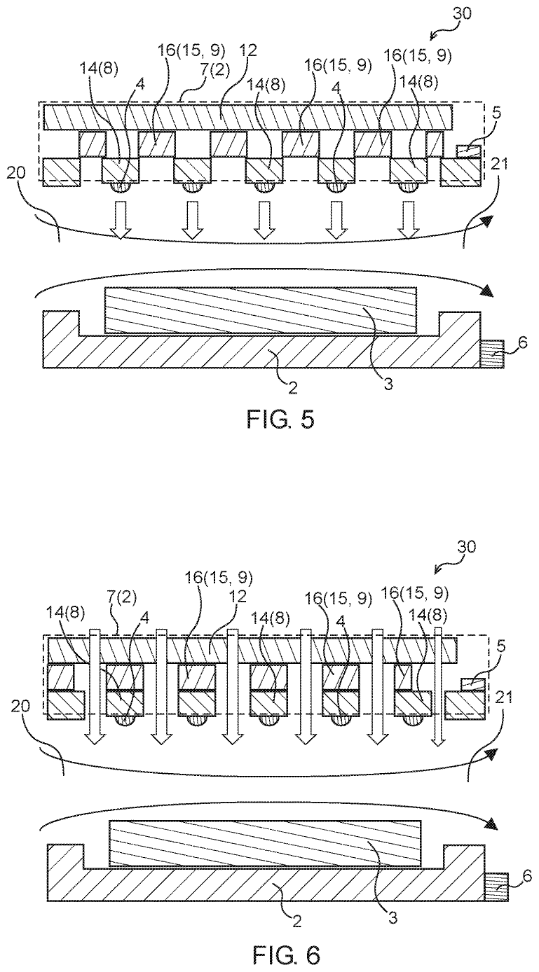

FIG. 5 is a schematic cross-sectional view of a photocatalyst module 30 (window 7 is in the light-shielding state) according to a second modification of the first embodiment, and corresponds to FIGS. 1 and 3. FIG. 6 is a schematic cross-sectional view of the photocatalyst module 30 (window 7 is in the light-collecting state) according to the second modification of the first embodiment, and corresponds to FIGS. 2 and 4.

In the second modification, the light-shielding member 9 is a second grid 15 including a plurality of second rods 16, and the window 7 includes a plate-shaped translucent member 12 outside the light-shielding member 9. The other configurations are the same as those of the first embodiment and the first modification described above.

The material of the translucent member 12 is, for example, glass or a translucent resin. Since the window 7 has the translucent member 12, it is possible to prevent air from flowing into the housing 2 through the window 7 even when the window 7 is brought into the light-collecting state as illustrated in FIG. 6. This can prevent the adhesion of dust flowing into the housing 2 together with air to the photocatalyst portion 3 or the disturbance of airflow inside the housing 2.

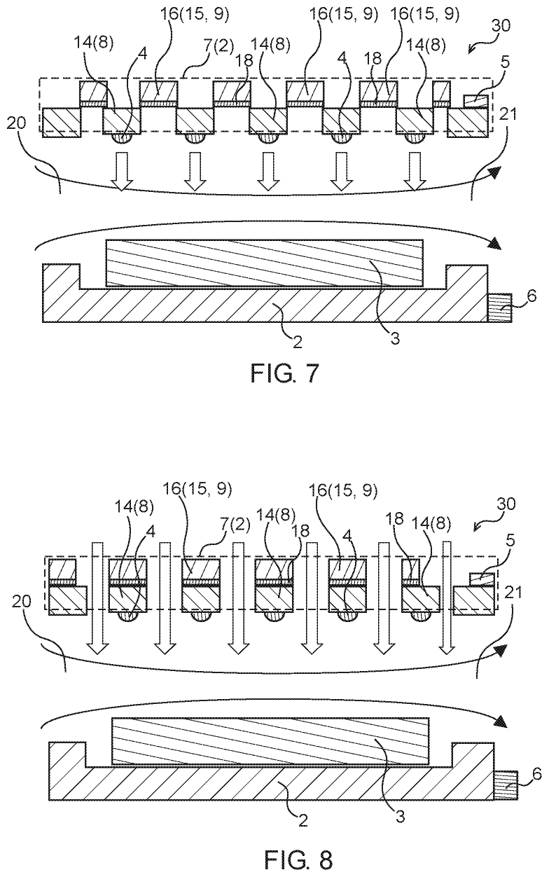

FIG. 7 is a schematic cross-sectional view of a photocatalyst module 30 (window 7 is in the light-shielding state) according to a third modification of the first embodiment, and corresponds to FIGS. 1, 3, and 5. FIG. 8 is a schematic cross-sectional view of the photocatalyst module 30 (window 7 is in the light-collecting state) according to the third modification of the first embodiment, and corresponds to FIGS. 2, 4, and 6.

In the third modification, the light-shielding member 9 is a second grid 15 including a plurality of second rods 16, and the second rods 16 include mirrors 18 provided to reflect reflection light resulting from light emitted to the photocatalyst portion 3 from the light source unit 4. The other configurations are the same as those of the first embodiment and the first modification described above.

When the window 7 is in the light-shielding state as illustrated in FIG. 7, the openings of the first grid 8 are covered with the second rods 16 of the second grid 15. The mirrors 18 can be provided on the inner surfaces of the second rods 16. In this case, when light is emitted toward the photocatalyst portion 3 from the light-emitting elements provided on the first rods 14 of the first grid 8, light is reflected on the inner surfaces of the photocatalyst portion 3 and the housing 2 and returns to the window 7. The reflected light is further reflected on the mirrors 18 and emitted to the photocatalyst portion 3. In this manner, light can be repeatedly emitted to the photocatalyst portion 3, and thus, photocatalytic activity of the photocatalyst portion 3 can be improved.

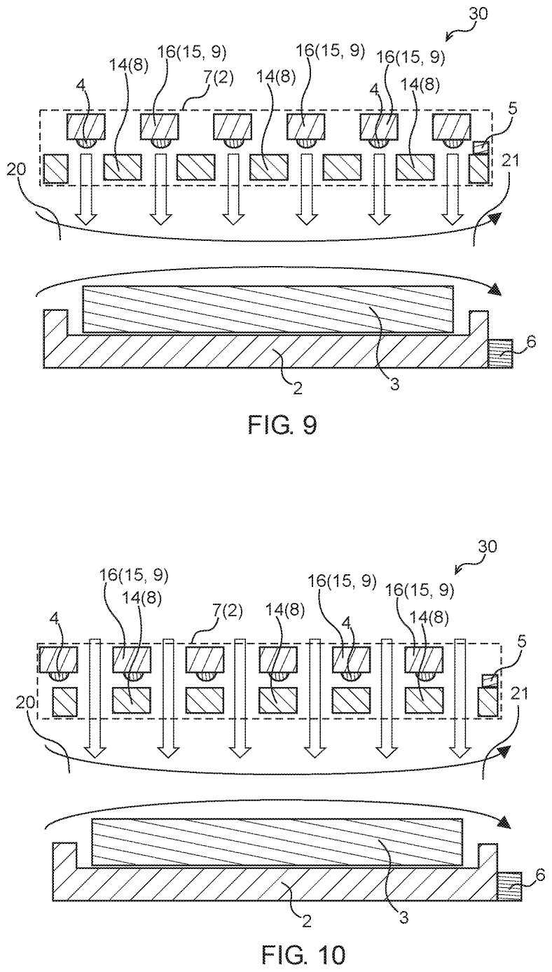

FIG. 9 is a schematic cross-sectional view of a photocatalyst module 30 (window 7 is in the light-shielding state) according to a fourth modification of the first embodiment, and corresponds to FIGS. 1, 3, 5, and 7. FIG. 10 is a schematic cross-sectional view of the photocatalyst module 30 (window 7 is in the light-collecting state) according to the fourth modification of the first embodiment, and corresponds to FIGS. 2, 4, 6, and 8.

In the fourth modification, the light-emitting elements of the light source unit 4 are arranged on the plurality of second rods 16 constituting the second grid 15 (light-shielding member 9). Further, the light-emitting elements can be arranged at intervals on each of the second rods 16. Thus, the entire photocatalyst portion 3 can be efficiently irradiated with light. The interval between the light-emitting elements is 5 cm or more and 20 cm or less, for example.

### Second Embodiment

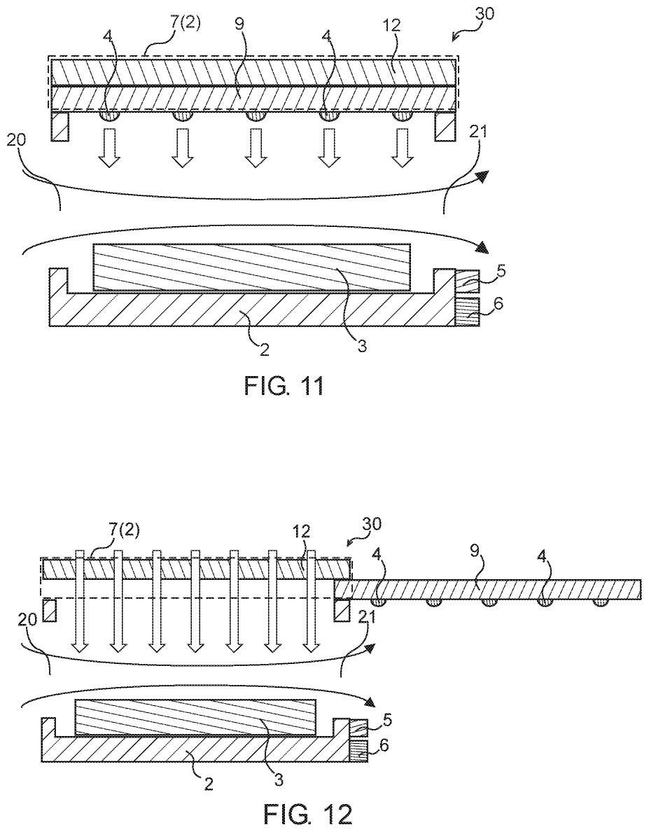

FIG. 11 is a schematic cross-sectional view of a photocatalyst module (light-shielding state) according to a second embodiment, and FIG. 12 is a schematic cross-sectional view of the photocatalyst module (light-collecting state) according to the second embodiment.

In the second embodiment, in the light-shielding state, a window 7 has a structure obtained by stacking a plate-shaped light-shielding member 9 and a plate-shaped translucent member 12 disposed outside the light-shielding member 9. Further, a plurality of light-emitting elements included in a light source unit 4 are arranged on the inner surface of the light-shielding member 9. The plurality of light-emitting elements are arranged so as to be capable of irradiating the entire photocatalyst portion 3 with light. The interval between the light-emitting elements is, for example, 5 cm or more and 20 cm or less. The plurality of light-emitting elements can also be arranged in a matrix. The other configurations are the same as those of the first embodiment.

When it is relatively dark outside the housing 2, the light-shielding member 9 and the translucent member 12 are stacked as illustrated in FIG. 11, and thus, the window 7 is in the light-shielding state. In this case, a control unit 6 turns on the light source unit 4 to irradiate the photocatalyst portion 3 with light. Thus, the photocatalyst portion 3 can exhibit photocatalytic activity and can decompose and remove organic substances in air flowing through an inlet 20. In addition, since the window 7 is in the light-shielding state, the leakage of light inside the housing 2 can be prevented.

When the outside of the housing 2 becomes bright and the output from an illuminance sensor 5 exceeds a threshold value, the control unit 6 turns off the light source unit 4 and moves the light-shielding member 9, using a switching mechanism, so as to irradiate the photocatalyst portion 3 with external light transmitting through the translucent member 12. For example, the control unit 6 changes the photocatalyst module 30 illustrated in FIG. 11 into the photocatalyst module 30 illustrated in FIG. 12.

In this case, the external light is introduced into the housing through the translucent member 12, and the photocatalyst portion 3 is irradiated with the introduced external light. Thus, the photocatalyst portion 3 can exhibit photocatalytic activity, whereby organic substances in the air flowing through the inlet 20 can be decomposed and removed.

Further, the description of the first embodiment applies to the second embodiment as long as there is no inconsistency.

### Third Embodiment

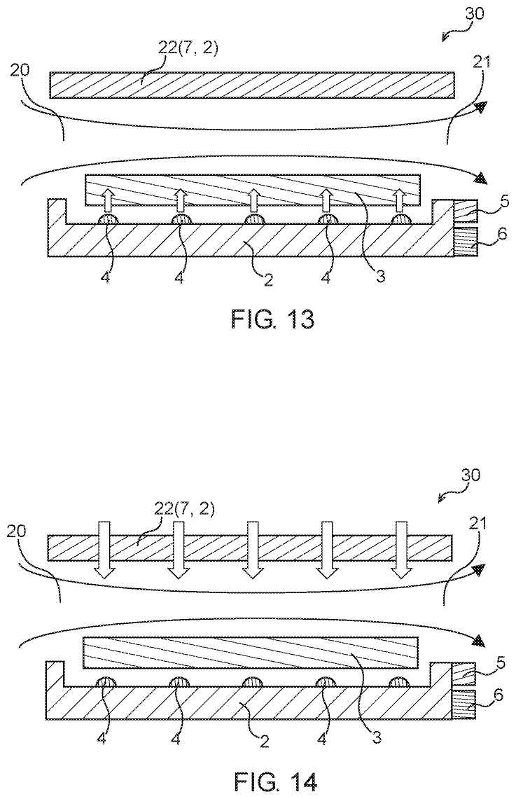

FIG. 13 is a schematic cross-sectional view of a photocatalyst module (light-shielding state) according to a third embodiment, and FIG. 14 is a schematic cross-sectional view of the photocatalyst module (light-collecting state) according to the third embodiment.

In the third embodiment, a window 7 includes an optical shutter 22. The optical shutter 22 is, for example, a liquid crystal optical shutter or a mechanical optical shutter (e.g., louver shutter). The optical shutter 22 is connected to a control unit 6 so that the optical shutter 22 can be opened and closed by the control unit 6.

In addition, a plurality of light-emitting elements included in a light source unit 4 are arranged on a housing 2 below a photocatalyst portion 3 (on the side opposite to the window 7). The plurality of light-emitting elements are arranged so as to be capable of emitting light toward the entire photocatalyst portion 3. The interval between the light-emitting elements is, for example, 5 cm or more and 20 cm or less. The plurality of light-emitting elements can also be arranged in a matrix. The other configurations are the same as those of the first embodiment.

When it is relatively dark outside the housing 2, the optical shutter 22 is closed, so that the window 7 is in the light-shielding state. In this case, the control unit 6 turns on the light source unit 4 to irradiate the photocatalyst portion 3 with light as illustrated in FIG. 13. Thus, the photocatalyst portion 3 can exhibit photocatalytic activity, whereby organic substances in the air flowing through an inlet 20 can be decomposed and removed. In addition, since the window 7 is in the light-shielding state, the leakage of light inside the housing 2 can be prevented.

When the outside of the housing 2 becomes bright and the output from an illuminance sensor 5 exceeds a threshold value, the control unit 6 turns off the light source unit 4 or decreases the amount of light from the light source unit 4 and switches the optical shutter 22 to an open state to bring the window 7 into the light-collecting state. In this case, as illustrated in FIG. 14, external light is introduced into the housing through the optical shutter 22, and the photocatalyst portion 3 is irradiated with the introduced external light. Thus, the photocatalyst portion 3 can exhibit photocatalytic activity, whereby organic substances in the air flowing through the inlet 20 can be decomposed and removed.

Further, the description of the first embodiment applies to the third embodiment as long as there is no inconsistency.

1. A photocatalyst module comprising: a housing; a photocatalyst portion disposed inside the housing; a light source unit provided to irradiate the photocatalyst portion with light; an illuminance sensor provided to detect brightness outside the housing; and a control unit, wherein
the housing is provided with a window, and
the control unit is provided to switch the window between a light-shielding state for shielding external light entering the housing and a light-collecting state for allowing external light to enter the housing on the basis of an output from the illuminance sensor.

2. The photocatalyst module according to claim 1, wherein the control unit is provided in such a manner that, when the window is in the light-shielding state, the light source unit irradiates the photocatalyst portion with light, and when the window is in the light-collecting state, the light source unit is turned off or an amount of light emitted from the light source unit is reduced.

3. The photocatalyst module according to claim 1, wherein
the housing comprises an inlet and an outlet, and
the photocatalyst portion is placed at a position where a gas flowing from the inlet to the outlet comes into contact with the photocatalyst portion.

4. The photocatalyst module according to claim 1, wherein
the window comprises a light-shielding member and a first grid having a plurality of openings, and
the control unit is provided to switch the window between the light-collecting state and the light-shielding state by moving the light-shielding member on the basis of the output from the illuminance sensor, wherein the light-shielding member does not cover the openings in the light-collecting state, and the light-shielding member covers the openings in the light-shielding state.

5. The photocatalyst module according to claim 4, wherein the window comprises a translucent member that covers the first grid.

6. The photocatalyst module according to claim 4, wherein
the first grid comprises a plurality of first rods,
each of the openings is an opening between two first rods adjacent to each other, and
the light source unit is provided to emit light to the photocatalyst portion from the first rods.

7. The photocatalyst module according to claim 4, wherein
the light-shielding member is a second grid having a plurality of second rods, and
the control unit is provided to switch the window between the light-collecting state and the light-shielding state by moving the light-shielding member on the basis of the output from the illuminance sensor, wherein the second rods do not cover the openings in the light-collecting state, and the second rods cover the openings in the light-shielding state.

8. The photocatalyst module according to claim 7, wherein
the second rods comprise a mirror provided to reflect reflection light resulting from light emitted to the photocatalyst portion from the light source unit.

9. The photocatalyst module according to claim 7, wherein the light source unit is provided to emit light to the photocatalyst portion from the second rods.

10. The photocatalyst module according to claim 1, wherein
the window comprises a translucent member and a light-shielding member, and
the control unit is provided to switch the window between the light-collecting state and the light-shielding state by moving the light-shielding member on the basis of the output from the illuminance sensor, wherein the light-shielding member does not cover the translucent member in the light-collecting state, and the light-shielding member covers the translucent member in the light-shielding state.

11. The photocatalyst module according to claim 10, wherein the light source unit is provided to emit light to the photocatalyst portion from the light-shielding member.

12. The photocatalyst module according to claim 1, wherein the window comprises an optical shutter.
